(19) United States
(12) Patent Application Publication
(10) Pub. No.: US 2025/0305022 A1
(43) Pub. Date: Oct. 2, 2025

(54) METHODS OF SCREENING FOR ANTIBIOTICS

(71) Applicant: Research Foundation of the City University of New York, New York, NY (US)

(72) Inventors: Haiping Cheng, Mt. Kisco, NY (US); Ertan Kastrat, Yonkers, NY (US)

(21) Appl. No.: 19/092,938

(22) Filed: Mar. 27, 2025

Related U.S. Application Data
(60) Provisional application No. 63/571,737, filed on Mar. 29, 2024.

(51) Int. Cl. C12Q 1/18

(52) U.S. Cl. CPC C12Q 1/18

(57) ABSTRACT

A method for screening for an antibiotic. A first bacterial sample is streaked to form an internal space that is surrounded by the streak. The streak is permitted to grow for at least 1 day. A second bacterial sample is spotted within the internal space and at least two days of growth time is permitted. A sample between the spotted location and streaked location is harvested and an antibiotic is harvested therefrom.

### CROSS-REFERENCE TO RELATED APPLICATIONS

This application claims priority to, and is a non-provisional of, U.S. Patent Application 63/571,737 (filed Mar. 29, 2024), the entirety of which is incorporated herein by reference.

### STATEMENT OF FEDERALLY SPONSORED RESEARCH OR DEVELOPMENT

This invention was made with government support under grant number GM081147 awarded by the National Institute of Health. The government has certain rights in the invention.

### BACKGROUND OF THE INVENTION

The subject matter disclosed herein relates to methods of isolating broad spectrum antibiotics. Bacteria are known to produce compounds to inhibit the growth of microbial competitors in their environment. This inhibition is typically mediated through the production of broad-spectrum secondary metabolites and narrow-spectrum peptides, such as bacteriocins and microcins, aiding bacteria in their competition against other species and strains. Border formation between colonies is a peculiar type of competition, where bacteria within a colony collectively inhibit the growth and expansion of an encroaching neighboring colony, resulting in the formation of a cell-free or low cell density border between the colonies. Inter-strain inhibition between swarming bacterial colonies has been well described, using terms such as border formation, kin discrimination and/or recognition, mutual inhibition, and demarcation line formation, to describe the formation of an inhibitory boundary observed between encroaching colonies of Bacillus subtilis, E. coli, Pseudomonas aeruginosa, Proteus mirabilis, and Myxococcus xanthus, mediated by cell-contact dependent killing and cell surface receptors involved in distinguishing strain identities.

One of the reasons proposed to explain the lack of progress towards novel antibiotic discovery over the past few decades is the idea that antibiotics have been overmined from culturable microorganisms. Though drug discovery using traditional screening methods has slowed, these methods remain a viable option allowing for screening for bacterial competitive interactions that may lead to novel drug discoveries. The difficulty in finding new antibiotic compounds has sparked the development of novel and complex techniques, such as culturing classically unculturable bacteria, high-throughput screening of drug libraries, activation of silent biosynthetic gene clusters, and artificial intelligence guided drug design, to screen for new drug candidates. While each of these methods are viable strategies, there remains a need to addition screening methods for antibiotics.

The discussion above is merely provided for general background information and is not intended to be used as an aid in determining the scope of the claimed subject matter.

### SUMMARY

This disclosure provides a method for screening for an antibiotic. A first bacterial sample is streaked to form an internal space that is surrounded by the streak. The streak is permitted to grow for at least 1 day. A second bacterial sample is spotted within the internal space and at least two days of growth time is permitted. A sample between the spotted location and streaked location is harvested and an antibiotic is harvested therefrom.

In a first embodiment, a method of screening for an antibiotic is provided. The method comprising sequential steps of: streaking a first bacterial sample to form surrounded internal space on a supporting media, thereby forming an initial streak; waiting at least 1 day; spotting a second bacterial sample on the supporting media in a center of the initial streak, thereby defining a spot location; waiting at least 2 days; harvesting a sample at a location between the spot location and an edge of the initial streak, thereby producing a harvested sample; and isolating an antibiotic from the harvested bacteria sample.

In a second embodiment, a method of screening for an antibiotic is provided. The method comprising sequential steps of: streaking a first bacteria sample in a pattern on a supporting media, wherein the pattern has a pattern edge; waiting at least 1 day; spotting a second bacterial sample on the supporting media at a distance of at least 0.5 cm but less than 2.0 cm from an edge of the pattern edge; waiting at least 2 days; harvesting a sample at a location between the spot location and the pattern edge, thereby producing a harvested sample; and isolating an antibiotic from the harvested bacteria sample.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 depicts one example of a method for screening for an antibiotic.

FIG. 2 depicts formation of a low cell density border between clonal colony fronts during swimming motility.

FIG. 3A and FIG. 3B show that the observed inhibitory effect is independent of cell contact, wherein FIG. 3A shows controls and FIG. 3B shows experimental growth patterns.

FIG. 4A, FIG. 4B and FIG. 4C depict cultures of S. aureus, A. baumannii and K. pneumoniae under select conditions.

FIG. 5A and FIG. 5B depict cultures of ΔluxS and ΔrpoS Keio mutants from E. coli under select conditions.

FIG. 6 shows a graph of CFU per mL for a saline agar (control) and a E. coli BW25113 culture with 1-day and 2-day preincubation periods.

FIG. 7A is a fluorescence image of an E. coli BW25113 culture as a control.

FIG. 7B is a fluorescence image of an E. coli BW25113 culture center spot of a 1-day preincubation within a square streak. FIG. 7C is a graph that quantifies the degree of fluorescence.

FIG. 8 shows different strains bacteria were spotted, some of which included multi-drug resistant clinical isolates, in the center region of each grid.

### DETAILED DESCRIPTION OF THE INVENTION

This disclosure provides a simple method to screen bacteria for the production of inhibitory compounds which may not be detectable using typical screening methods. As detailed below, some of the most well-studied organisms in bacteriology are producing inhibitory compounds not previously described. Surprisingly, the antibiotics are generated through intra-species exposure.

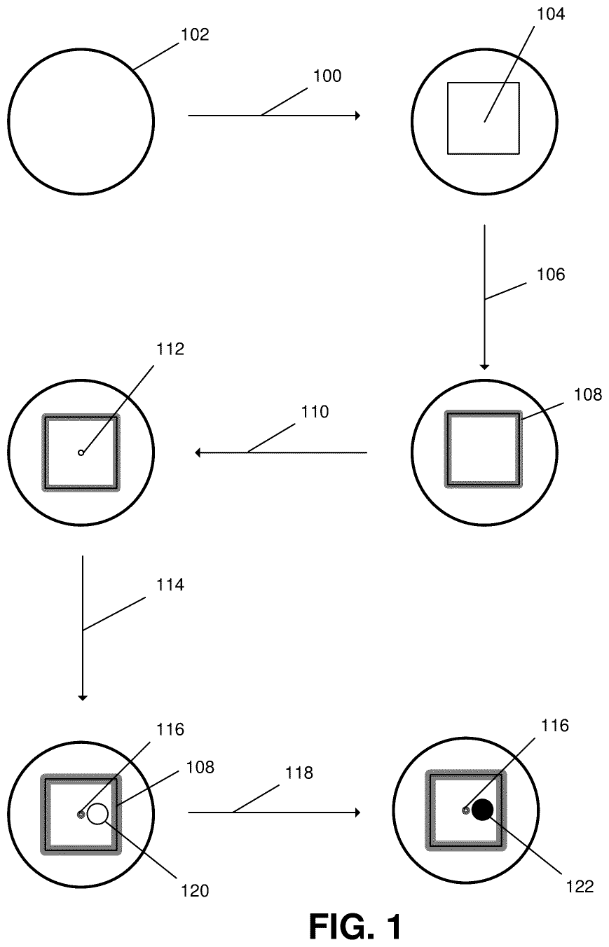

Referring to FIG. 1, in step 100, an ager-filled plate 102 is initially streaked with a first bacterial sample to form a surrounded internal space 104. The streaking may be in a variety of patterns such as, for example, circular or square. In the embodiment of FIG. 1, the pattern is a square pattern. The ager is generally at a concentration between 0.3% to 1.5% by weight. In step 106, the individual waits at least 1 day for the first bacterial sample to grow to produce a grown initial streak 108. Antibiotics are secreted from the grown initial streak 108. The surrounded internal space 104 has a particular high concentration of the antibiotic (due to the secretions coming from multiple directions) with a higher concentration near edges of the grown initial streak 108. In some embodiments, a wait time of 1-5 days, 1-4 days, 1-3 days 1-2 days or 1 day is observed before proceeding to step 110. In step 110, a second bacteria sample is spotted in a center 112 of the internal space 108, wherein the second bacteria sample is the same species as the first bacteria sample. In some embodiments, the first and second bacteria samples are the same species and the same strain. In step 114, the individual waits at least 1 day for the second bacterial sample to grow to produce a grown bacterial sample 116. Additionally, further growth of the grown initial streak 108 may also occur during step 114. In step 118, a sample is harvested from a location 120 that is between the grown bacterial sample 116 and the grown initial streak 108. In one embodiment, the sample is a sample of the supporting media such that the harvesting produces an excavated cavity 122 in the supporting media. Examples of supporting media include agar, agarose, artificial gels or a porous material (e.g. a porous sponge) with liquid growth medium. An antibiotic is then isolated from the harvested supporting media sample using conventional chemical methodologies. For example, the liquid growth medium may be harvested from the sponge, and the liquid subsequently removed, thereby isolate the dissolved antibiotic. Additional details follow.

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Initial formation of an inhibitory border between swimming clonal inoculants: Initially, an inhibitory border forming between swimming bacterial colony fronts expanding through 0.3% motility agar was first serendipitously seen with Sinorhizobium meliloti Rm1021. An experiment was being conducted to screen for motility in S. meliloti mutants and had left Petri dishes on the bench at room temperature for some weeks. Upon inspection, the swimming colonies had formed a visible boundary with their neighboring colonies. The expected radial expansion of the colonies did not occur—the colonies formed a square shape. The inhibitory border (see location 120 of FIG. 1) was not cell-free, but rather harbored a lower cell density compared to regions where opposing colony fronts did not meet.

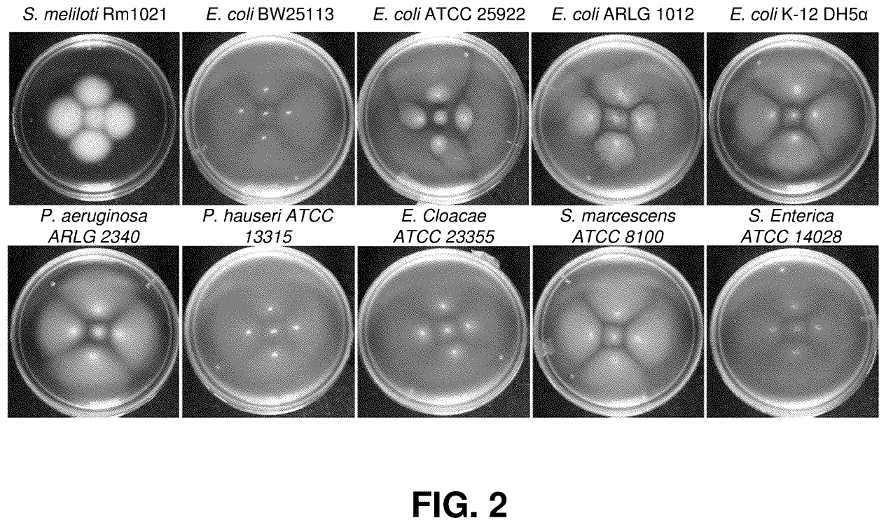

Based on this observation, further experiments were conducted to screen for the prevalence and ubiquity of intra-strain inhibition between swimming colonies of flagellated bacteria (Table 1) using S. meliloti, four strains of E. coli, Pseudomonas aeruginosa, Proteus hauseri, Enterobacter cloacae, Serratia marcescens, and Salmonella enterica, to reproduce the initial observations, with some modification. The flagellated bacteria were inoculated in 0.3% motility agar in a quincunx pattern to visually exacerbate inhibitory border formation (FIG. 2). This pattern was chosen to exacerbate the inhibitory effect on the center inoculant, leading to the formation of a deformed square-shaped colony with smaller size than the surrounding colonies. All five inoculation points used bacteria from the same colony to account for genetic variation between inoculants. All bacteria produced an inhibitory boundary when challenged with a neighboring colony. The degree of inhibition appears to vary, as some bacteria display inter-colony borders composed of lower cell density, while in other cases, the borders appear less pronounced due to an increased concentration of cells in the region. The most prominent inhibitory effect is seen with the center inoculant, as its colony is inhibited on four fronts, resulting in a swimming colony with a square shape. Inoculating bacteria in low nutrient media enhanced the appearance of the inhibitory border compared to rich media (data not shown), though this was not required for border formation. This data suggest there is a common mechanism of intra-strain inhibition in bacteria.

FIG. 2 depicts formation of a low cell density border between clonal colony fronts during swimming motility. Bacteria from the same colony were inoculated at 5 points in media containing 0.3% agar and incubated for 24 h at 37° C., except for S. meliloti, which was incubated for 48 h at 30° C. All strains were inoculated in low nutrient media consisting of 10% Luria-Bertani (LB) agar (0.1% tryptone and 0.05% yeast extract), except for S. meliloti, which was inoculated in the nutrient rich medium, LBMC (LB agar supplemented with magnesium and calcium). An inhibitory border between colony fronts is observed, with the strongest effect seen with the center inoculant. Cells were inoculated in a quincunx pattern with each point being 1 cm from the center inoculant.

TABLE 1
Strains used

| Bacteria | Key characteristics | Source or Reference |
|---|---|---|
| iAcinetobacter | MDR clinical isolate. Resistant to: | ARLG* |
| baumannii ARLG | Amikacin, Amp-sulb, Aztreonam, | |
| 1783 | Cefepime, Ceftazidime, Cipro, | |
| | Doripenem, Gentamicin, Imipenem, | |
| | Levofloxacin, Meropenem, Pip-tazo, | |
| | Ticarcillin/clavulanate, Tobramycin, | |
| | Trimethoprim/Sulfamethoxazole | |
| | (SXT) | |
| Enterobacter cloacae | Cephalosporinase + | ATCC** |
| ATCC 23355 | | |
| Escherichia coli | MDR clinical isolate. Resistant to: | ARLG |
| ARLG 1012 | Amikacin, Amox-clav, Amp-sulb, | |
| | Ampicillin, Aztreonam, Cefazolin, | |
| | Cefepime, Cefotaxime, Cefotetan, | |
| | Cefoxitin, Ceftazidime, Ceftriaxone, | |
| | Cefuroxime, Cephalothin, Cipro, | |
| | Ertapenem, Gentamicin, Imipenem, | |
| | Levo, Meropenem, Moxi, Pip-tazo, | |
| | Tetracycline, Ticar-clav, | |
| | Tobramycin, SXT, Doripenem | |
| Escherichia coli | Parent strain for Keio knockout | CGSC*** |
| BW25113 | collection | |
| Escherichia coli | rpoS746(del)::kan | CGSC |
| JW5437-1 ΔrpoS | Keio knockout collection. | |
| Escherichia coli | luxS768(del)::kan | CGSC |
| JW2662-1 ΔluxS | Keio knockout collection. | |
| Escherichia coli DH5α | High transformation efficiency; | FEMS Microbiol. |
| | derived from K-12 strain. | Rev. 41, 19-33 |
| | | (2017) |
| Escherichia coli | Uropathogenic clinical isolate. | ATCC |
| ATCC 25922 | Reference strain for MIC testing. | |
| Klebsiella pneumoniae | MDR clinical isolate. Resistant to: | ARLG |
| ARLG 1002 | Amikacin, Amox-clav, Amp-sulb, | |
| | Ampicillin, Aztreonam, Cefazolin, | |
| | Cefepime, Cefotaxime, Cefotetan, | |
| | Cefoxitin, Ceftazidime, Ceftriaxone, | |
| | Cefuroxime, Cephalothin, Cipro, | |
| | Ertapenem, Gentamicin, Imipenem, | |
| | Meropenem, Nitrofur, Pip-tazo, | |
| | Ticar-clav, Tobramycin, SXT, | |
| | Doripenem | |
| Pseudomonas | MDR clinical isolate. Resistant to: | ARLG |
| aeruginosa ARLG | Amikacin, Aztreonam, Cefepime, | |
| 2340 | Ceftazidime/Avibactam/Tazobactam, | |
| | Ciprofloxacin, Doripenem, | |
| | Gentamicin, Imipenem, | |
| | Levofloxacin, Meropenem, | |
| | Piperacillin-Tazobactam, Ticarcillin- | |
| | Clavulanate, Tobramycin | |
| Pseudomonas | Strain: Boston 41501 | ATCC |
| aeruginosa | | |
| ATCC 27853 | | |
| Proteus hauseri ATCC | Originally deposited as P. vulgaris | ATCC |
| 13315 | | |
| Salmonella enterica | Subspecies enterica serovar | ATCC |
| ATCC 14028 | Typhimurium | |
| Serratia marcescens | Non-pigmented | ATCC |
| ATCC 8100 | | |
| Sinorhizobium meliloti | Wild type strain | J Appl Microbiol. |
| Rm1021 | | 1997 May;82(5): |
| | | 610-4 |
| Staphylococcus aureus | MDR clinical isolate. Resistant to: | ARLG |
| ARLG 1574 | | |
| | Ciprofloxacin, Clindamycin, | |
| | Erythromycin, Tetracycline | |
| Staphylococcus aureus ATCC 25923 | Strain: Seattle 1945 | ATCC |

*ARLG (Antibiotic Resistance Leadership Group)
**ATCC (American Type Culture Collection)
***CGSC (Coli Genetic Stock Center, Yale University)

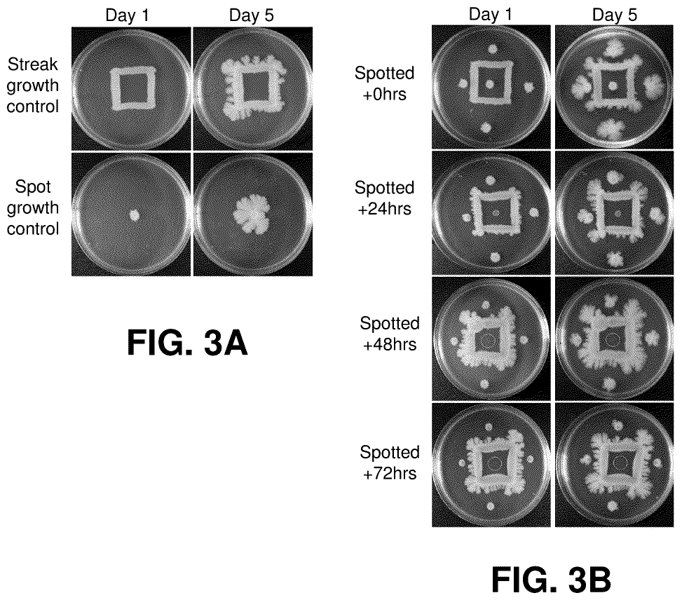

Inhibition is not cell-contact dependent and is enhanced by the age of bacterial colonies: With reference to FIG. 3A and FIG. 3B, to assess if the observed inhibition was the result of a compound released by bacteria as opposed to being a form of cell-contact dependent inhibition, the swimming inoculation pattern was modified by spotting and square streaking bacteria onto LB containing 0.8% agar to determine if intra-strain inhibition can occur across a distance. The bacteria were inoculated in a square streak pattern to represent the four surrounding inoculants from the swimming assays. A spotting pattern was chosen to represent the center inoculant from the previous assay, allowing inoculation in the center of and outside the square streak to compare the degree of inhibition at different positions. Bacteria spotted in the center are exposed to the highest concentration of compounds secreted by surrounding bacteria due to multiple merging diffusion fronts of the surrounding square streak. This concept is similar to placing multiple antibiotic infused paper disks onto the agar in a similar pattern and observing stronger inhibition in regions where diffusion fronts of the drugs combine and intersect. Five spotted inoculants were added at the same time (+0 h) as the square streak was produced, or added after 24, 48, or 72 h following initial incubation of the square streak, to assess the effect of colony age on the degree of inhibition. If inhibition is mediated by a diffusible compound, incubation of the square streak prior to spotting the bacteria would result in a stronger inhibitory effect as the inhibitor would accumulate in the growth medium over time. Four spot inoculants were added near each outer edge of the square streak (left, top, right, bottom; 0.5 cm, 1.0 cm, 1.5 cm, 2.0 cm from the square streak's outer edge, respectively), with the fifth spotted in the center of the square streak, 2.0 cm from the square streak's inner edge. Any variation in degree of inhibition between the center and surrounding inoculants could then be attributed to the merging of diffusion fronts, which may carry an inhibitory compound, resulting in a stronger inhibitory effect.

As controls, a square streak was produced or a single spot inoculant of E. coli BW25113 was placed onto LB 0.8% agar (see FIG. 3A). FIG. 3A and FIG. 3B show that the observed inhibitory effect is independent of cell contact. Escherichia coli BW25113 was streaked in a square pattern and spotted on LB containing 0.8% agar. Cells streaked in a square pattern exhibit outward growth but no inward expansion (FIG. 3A, top row). The spot inoculant grows outward, uninhibited (FIG. 3A, bottom row). Images were taken at Day 1 and Day 5 of incubation. The two inoculation methods were combined by incubating the square streak for 0, 24, 48, or 72 h, followed by spotting an inoculant on five points at a 0.5 cm increment, beginning from the left of the square streak, going clockwise distance (0.5 cm on the left to 2.0 cm below the square streak).

In FIG. 3B, E. coli was spotted at the same time as the square streak was produced (FIG. 3B, Top row). All five spotted colonies initially appeared to grow to a similar density after 1 day of incubation. However, the surrounding inoculants continued to grow over a 5-day period while the center inoculant's growth was inhibited after the first day of incubation. Additionally, bacteria spotted outside the square streak preferentially grew away from the neighboring square streak; this effect was slightly reduced as distance between the two inoculants increased. Preincubation of the square streak for 24, 48, or 72 h prior to spotting resulted in surrounding spotted colonies that were similar in size compared to those inoculated simultaneously with the square streak for the first day of incubation. However, deviation in size of surrounding spotted inoculants at the end of their 5-day incubation period was observed, where colonies spotted at earlier timepoints were larger than those spotted after preincubation of the square streak (FIG. 3B, descending rows, Day 5 column). In comparison to the surrounding inoculants, the center inoculant's growth was inhibited to a greater degree as prior incubation time of the square streak increased (FIG. 3B, descending rows). No growth was visually observable with the center inoculant when the square streak was preincubated for 72 h (FIG. 3B, bottom row). In all cases, the center inoculant does not appear to grow past the first day of incubation, while the surrounding spotted inoculants continue to grow. This suggests inhibition is not merely due to nutrient depletion, as bacteria can continue to grow, and it is likely an inhibitory compound is accumulating in the growth medium over time, leading to regulation of total biomass on the growth medium. Longer preincubation of the square streak results in stronger inhibition of the spotted inoculants, with the stronger effect seen with the center inoculant. Central inoculant spotting location highlighted for clarity (FIG. 3B, circles drawn on 48- and 72-h images).

Together, these data suggest inhibition is mediated by a diffusible compound and that concentration of the inhibitor increases over time. These data also suggest nutrient limitation is unlikely the sole cause of growth inhibition, as even with 8 days of total incubation time (72 h preincubation of the square streak followed by 5 additional days of growth, FIG. 3B, bottom row), surrounding colonies continue to grow while no growth is observed with the center inoculant following 1 day of incubation.

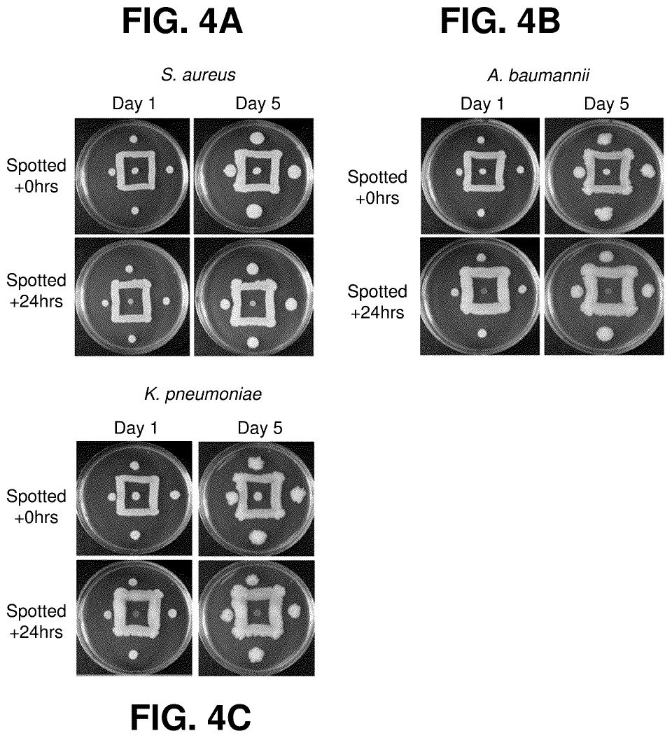

Non-flagellated bacteria also exhibit intra-strain inhibition: Intra-strain inhibition between colonies of non-flagellated bacteria has not been previously described. To address this and to determine the ubiquity of this phenomenon, square streak and spotting inoculation patterns were repeated using non-flagellated bacteria. Multi-drug resistant clinical isolates of Staphylococcus aureus, Acinetobacter baumannii, and Klebsiella pneumoniae were used (Table 1). The same inhibition phenotype previously seen with flagellated bacteria with the non-flagellated bacteria were observed (FIG. 4A, FIG. 4B, FIG. 4C). Deformation of expanding colony fronts can be seen, most notably between the left spotted inoculant and the neighboring square streak, and consistent with previous results, the strongest inhibition was observed with the center-spotted inoculant (FIG. 4A, FIG. 4B FIG. 4C, Day 5). Together, these results suggest intra-strain inhibition may be ubiquitous, as was seen in flagellated, non-flagellated, Gram-positive, and Gram-negative bacteria. Additionally, these data suggest inhibition of growth and border formation is not dependent on the presence of flagella, as the same inhibition pattern appears when non-flagellated cells are used. These data do not rule out that deformation of colony expansion is a chemotactic response, as flagellar-independent motility, such as twitching, gliding, spreading, or darting, may at least be partially responsible for deviation from radial colony expansion. However, the strongest evidence of chemically mediated growth-inhibition is displayed by the growth inhibition of inoculants spotted in the center of a square streak. This suggests biophysical factors are unlikely to be the cause of the observed phenotype.

Referring again to FIG. 4A-4C, the depicted images show inhibition screening of non-flagellated bacteria. FIG. 4A shows non-flagellated, multi-drug resistant clinical isolates, S. aureus ARLG 1574, FIG. 4B shows A. baumannii ARLG 1783 and FIG. 4C shows K. pneumoniae ARLG 1002 all exhibited the same inhibition phenotype as E. coli BW25113. Inhibition of the center inoculant is strongest and displays no visible change in growth over a period of 5 days. Deformation of colony fronts between the square streak and spotted inoculants are visible and most obvious when observing the closest spotted inoculant (left of streak) at Day 5.

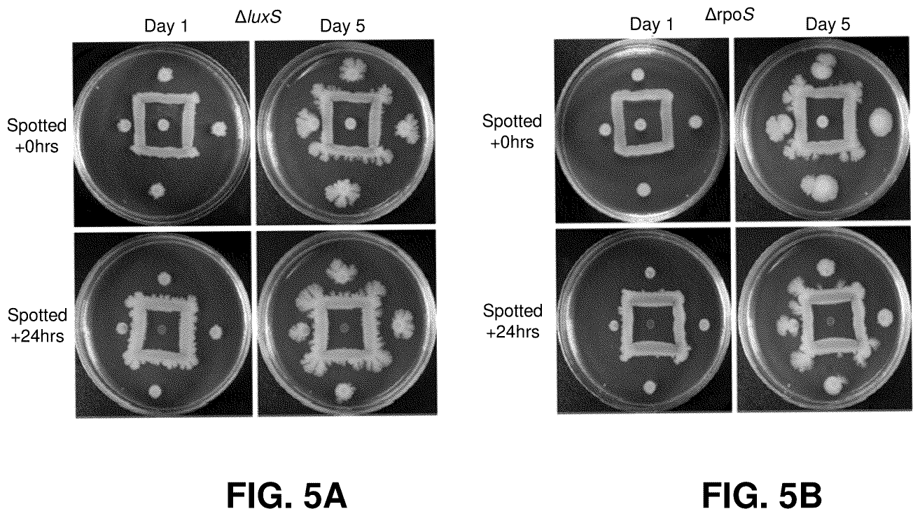

Inhibition phenotype is independent of quorum signaling and the stress response: Based on the previous observations showing a positive correlation between colony age and the strength of inhibition, transcriptional changes associated with quorum signaling or the stress response may play a role in the observed inhibition phenotype. Therefore, to assess if LuxS-mediated quorum signaling, or the RpoS-mediated stress response, were involved in the production of the inhibitory compound, further experiments were conducted. ΔluxS and ΔrpoS Keio collection mutants from E. coli BW25113 parent strain did not produce an altered phenotype when compared with the previous data (FIG. 5A and FIG. 5B). These results show the inhibition phenotype is independent of quorum signaling and the RpoS-mediated stress response. This suggests inhibitor production may occur at a constant basal rate or may require specific conditions to elicit upregulation and production of the active compound.

Referring again to FIG. 5A and FIG. 5B Inhibition is not regulated by quorum sensing or the stress response. Escherichia coli Keio collection knockouts in quorum sensing (ΔluxS, FIG. 5A) and the stress response (ΔrpoS, FIG. 5B) exhibit an identical inhibition phenotype as the parent strain BW25113, seen in FIG. 3A and FIG. 3B.

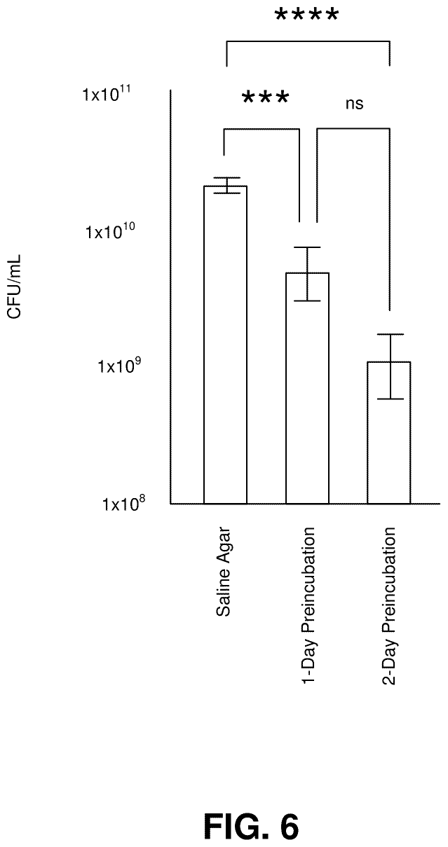

Growth inhibition is not due to nutrient depletion: To rule out the possibility of the observed growth inhibition resulting from nutrient depletion or starvation, E. coli BW25113 was spotted onto nutrient-free saline agar and compared CFU to bacteria spotted on nutrient rich media in the center of a 24-h and 48-h preincubated square streak. CFU 24 h was determined after incubation of the spotted inoculants and observed a reduction in CFU for inoculants added to the center of a streak preincubated for 1 day (P=0.0002) and 2 days (P<0.0001) (FIG. 6) compared to cells spotted onto nutrient-free saline. These data suggest that the inhibitory effect cannot be explained by a lack of nutrients and that the active compound may function through a bactericidal mechanism of action due to the reduction in viable bacterial cells.

Specifically, and with reference to FIG. 6, inhibition of growth for spotted center inoculant is not due to a starvation effect. Escherichia coli BW25113 was spotted on a saline agar plate, or on LB in the center of a square streak of E. coli BW25113 previously incubated for 1 or 2 days. Spotted inoculants were incubated for 24 h. Significant reduction in CFU is observed when spotted inoculants are added to plates containing the square bacterial streak (as seen in FIG. 3A and FIG. 3B) previously incubated for 1 (****P=0.0002) and 2 days (***P<0.0001). ns=not significant values are means from three replicates.

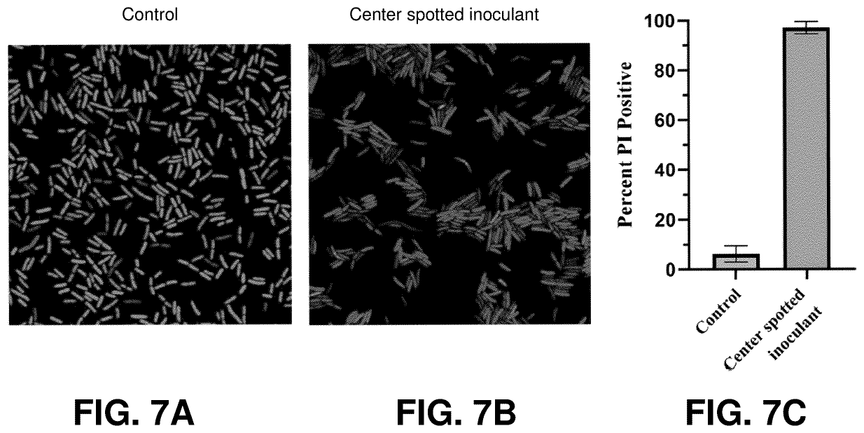

To gain preliminary insight into the mechanism of action for the inhibitory compound, E. coli BW25113 was stained with nucleic acid stains using cell permeable SYTO9 and cell impermeable Propidium Iodide (PI) to determine if membrane damage was occurring. The bacteria was spotted onto sterile LB agar as a control to compare with cells spotted in the center of a 24-h preincubated square streak, followed by 90 min of incubation prior to staining. Most cells spotted in the center of the square streak exhibited red fluorescence, indicating damage to the bacterial membrane (FIG. 7A, FIG. 7B, FIG. 7C). Taken together, these data suggest a diffusible compound is released by E. coli and causes cell death in bacteria of the same strain. Based on the previous observations, this mechanism is believed to be ubiquitous amongst bacteria.

The diffusible inhibitory compound caused damage to the bacterial cell membrane. Referring to FIG. 7A and FIG. 7B, Escherichia coli BW25113 was spotted onto an LB 0.8% agar (FIG. 7A, control) or in the center of a 1-day preincubated square streak of BW25113 (FIG. 7B). Plates were then incubated for 90 min and stained with cell permeable SYTO9 (green, live cells) and cell impermeable propidium iodide (PI, red cells, damaged membrane). Percentage of PI positive cells was determined from three independent fields of view from each condition (FIG. 7C).

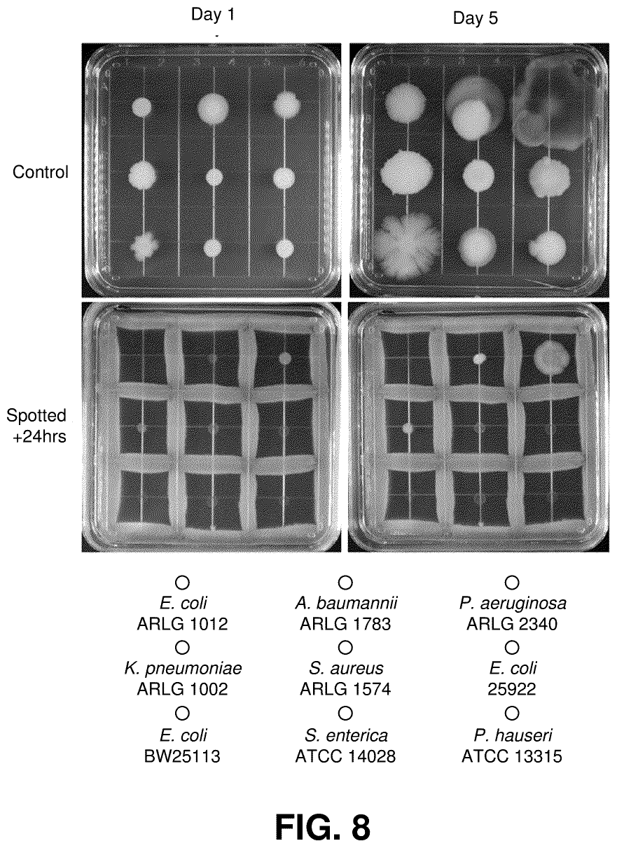

Escherichia coli inhibits the growth of other bacteria: Having demonstrated that intra-strain inhibition can be observed in diverse bacterial species, additional experiments were conducted to see if this inhibitory effect was limited to within a strain and if there could be a conserved mechanism in bacteria regulating this process. If this is a conserved mechanism common to all bacteria, cross-species inhibition may be observed. E. coli BW25113 was used as the producer strain for the inhibitory compound. E. coli was streaked in a 3×3 grid pattern and incubated the plate for 24 h. Following incubation, different strains of E. coli and other bacteria were spotted, some of which included multi-drug resistant clinical isolates (Table 1), in the center region of each grid (FIG. 8). As controls, these same bacteria were spotted onto a fresh LB agar to serve as a comparison for growth inhibition. Compared to controls, varying degrees of growth inhibition was observed against all spotted inoculants. These data suggest a diffusible inhibitory compound from E. coli has a broad spectrum of activity, affecting self and non-self. These data show E. coli can surprisingly inhibit the growth, to varying degrees, of other bacteria, suggesting a novel antibiotic compound could be derived from E. coli. These data show the disclosed simple and reproducible methods of screening can used to discover novel antibiotic agents from other bacteria. These data also reveal that one of the best-studied microorganisms, E. coli, can surprisingly exhibit broad-spectrum inhibitory activity against other bacteria.

Referring again to FIG. 8, inhibitory activity was observed across bacterial species. Escherichia coli BW25113 was streaked in a grid pattern and incubated for 24 h. Spotted inoculants, including multi-drug resistant clinical isolates, were added in-between E. coli streaks, incubated, and imaged for an additional 5 days with the streaked grid, or onto a plate with no grid streaked, as a control. Growth of all bacteria spotted onto the preincubated E. coli grid plate was inhibited to varying degrees.

Boundary formation in swimming E. coli colonies was previously described and found to be attributed to genetic variation within a population leading to inter-strain but not intra-strain boundary formation (Cell Reports, V 27, Issue 3, 16 Apr. 2019, pp 737-749). The results presented in this disclosure contrasts that finding, due to the observation of intra-strain boundary formation, even when using inoculants from a single colony (FIG. 2). Border formation due to inhibition between different swarming strains has been described for P. mirabilis, M. xanthus, P. aeruginosa, and B. subtilis, and is found to be cell-contact dependent. In this disclosure, border formation results from intra-strain inhibition independent of cell contact (FIG. 3A, FIG. 3B, FIG. 4A, FIG. 4B, FIG. 4C). Reports in the literature describe border formation between swarming P. dendritiformis colonies that was found to be mediated by a narrow-spectrum secreted protein, mediating intra-strain inhibition. In contrast, this disclosure is different from what has been reported in the literature, as cross-species and cross-strain inhibition (FIG. 8) is observed, the phenotype of which has not been described in the literature. The self-inhibition phenotype occurs over larger distance, contrasting with what has described for P. dendritiformis. Together, this suggests the observed phenotype is mediated by an undiscovered and undescribed compound.

Another report of intra-species boundary formation suggests the phenotype is due to a conserved genetic mechanism involved in controlling the flagellar response, contrasting with the observations of this disclosure, where inhibition occurs over a distance, even in non-flagellated bacteria (FIG. 3A, FIG. 3B, FIG. 4A, FIG. 4B and FIG. 4C). One study suggests border formation may be due to an inhibitory compound produced by bacteria and found that agar concentration and initial inoculation distance affect whether or not intra-strain boundary formation occurs, where agar concentrations <0.5% resulted in merging (Soft Matter, 2019, 15, 5400-5411). This contrasts with this disclosure of swimming inhibition in 0.3% agar but supports the conclusion that inhibition is mediated by a secreted compound (FIG. 2).

Without wishing to be bound to any particular theory, the variation of experimental conclusions amongst different groups could be due to an inhibitor being produced at low concentrations, resulting in a phenotype that is difficult to observe. The inhibitory compound may be produced at basal levels because inhibition is stronger with longer preincubation times of an initial streak (FIG. 3A and FIG. 3B). Basal-level transcription could explain the weak inhibitory border formation amongst swimming inoculants which traverse distances at a faster pace compared to distances traversed by surface inoculants. The slower spread of bacteria on the surface allows for accumulation of the inhibitory compound which can then exert its effects at a greater distance from the bacterial source. The disclosed inoculation patterns exacerbate the inhibition phenotype, possibly resulting in the merging of multiple inhibitor diffusion fronts, or, from overcrowding or exposure to the inhibitory compound leading to autoinduction of inhibitor production. Additionally, inhibitory activity is not dependent on quorum sensing nor the RpoS-mediated stress response (FIG. 5A and FIG. 5B), which are pathways known to induce antibiotic production. This suggests the active compound may be produced by a silent biosynthetic gene cluster which could be upregulated by finding the appropriate eliciting conditions. A compound with no obvious resistance in producer strains would likely only be secreted under specific conditions and at very low concentrations, otherwise, it would not allow for bacterial growth to initially occur.

Inhibition of growth was ruled out and being solely due to nutrient starvation by comparing CFU of bacteria spotted on nutrient-free agar against bacteria spotted in the center of a square streak (FIG. 6). Additionally, significant membrane damage was observed with SYTO9/PI staining of centrally spotted inoculants (FIG. 7A, FIG. 7B and FIG. 7C). A robust PI signal response used 90 min of exposure in the center streak. In the process of spotting cells, the inhibitory metabolite on the agar surface is likely is diluted when adding a liquid drop of bacteria. Then, a delay in a positive signal for membrane damage would be expected, as time would be needed for the compound to diffuse from the surrounding media into the diluted region.

The observed inhibition within and across bacterial strains and species may be the result of multiple compounds, or the inhibition may be due to a single compound produced by many bacteria utilizing a common biosynthetic pathway.

One aspect of the disclosed method involves excision of the inter-colony inhibitory region from the agar, followed by assessing conditions for chemical extraction and concentration of bioactive compounds contained within. The data suggest the inhibition-mediating compound may be produced at a low concentration, as the effect is only observable when using certain growth patterns.

The disclosed findings are surprising, as inhibition was observed within strains, across strains, and across species, and against multi-drug resistant clinical isolates, using E. coli BW25113 as the producer strain (FIG. 8). Escherichia coli, one of the most well-studied organisms, is not known to produce broad-spectrum antibiotic compounds. Escherichia coli does produce bacteriocins and microcins, which have a narrow spectrum of activity and are transcribed in conjunction with antitoxins, conferring resistance to producer strain. The observations suggest bacteria secrete a diffusible inhibitory compound into their surroundings which can limit the growth of clonal cells and other bacteria. This novel finding is in contrast to the protective effects E. coli can exhibit when cocultured with other bacterial species, as was shown with multi-species biofilms, where a protective effect is exerted between bacterial species against environmental insults. The difference in the observed killing effect versus the protective effect reported is believed to be due to the timing of coculturing, as the data suggests the time allotted for growth prior to introducing a new inoculant significantly alters the inhibition phenotype (FIG. 3B). Thus, cocultures do not allow for the accumulation of an inhibitory compound to biologically relevant levels. A higher density inoculum is used to significantly inhibit a fresh inoculum of far lower density, compared to an inoculum following 18 hours of growth. This idea holds true in the observation of inhibition at the lower cell density colony fronts seen in the swimming assays (FIG. 2) and with the spotted inoculants surrounding the square streak, where inhibition occurs at the intercolony fronts (FIGS. 2-4C). This may be due the inhibitor being expressed at a basal level, which can only be evidenced when a large number of bacteria, as seen in a colony, inhibits the growth of the low-density colony front, or of a freshly spotted inoculant. This reasoning justified as to why E. coli does not immediately self-inhibit when spotted onto a plate. Similarly, the density of bacteria per volume in culture compared to colonies on agar plates is orders of magnitude lower.

The disclosed method for screening for inhibitory activity amongst genetically diverse bacteria suggests self-inhibition is a widespread mechanism (FIGS. 2-4C). The data also suggest intra-strain competition may be an evolutionary conserved or convergent mechanism, as varying degrees of inhibition can be observed across species (FIG. 8), suggesting crosstalk between the pathways involved in mediating intra-strain competition. This disclosure shows the inhibition phenotype to be associated with membrane damage and that it is independent of nutrient availability, quorum sensing, and the RpoS-mediated stress response (FIGS. 5A-7). If the genes involved in inhibitor production are found and can be mutagenized or if the inhibitor can be neutralized, it could serve as a method to increase biomass and production for industrial fermentation. From a clinical perspective, identification of the active compound and mechanism of action provides fertile ground for the development of new drugs or identification of novel targets for antibiotics.

### Methods

### Culture Conditions and Media

LB (Luria-Bertani) medium was used for culturing all bacteria, except S. meliloti and S. aureus. LB supplemented with 2.5 mM MgSO4 and 2.5 mM CaCl2) (LBMC) was used for culturing S. meliloti. Trypticase Soy media was used for culturing S. aureus. Agar concentrations were 1.5% for solid medium, 0.3% for swimming assay, and 0.8% for surface inhibition assays. All strains from Table 1 were grown aerobically at 37° C., except for S. meliloti, which was similarly grown, at 30° C. Colonies for inoculation into broth were selected from freshly streaked plates and mid-logarithmic phase bacteria were subcultured into fresh medium at a concentration of 10⁷ CFU/mL. Liquid cultures were agitated at 200 rpm.

### Inhibition of Clonal Swimming Colonies

Bacterial cells were transferred to swimming plates in a pattern corresponding to the 4 vertices of a square 1 cm apart, with a 5th inoculant added to the center using a sterile toothpick in a 35 mm×10 mm Petri dish. Sinorhizobium meliloti was inoculated in LBMC with 0.3% agar. All other strains were inoculated in LB 0.3% agar with reduced concentrations of tryptone (0.1%) and yeast extract (0.05%). Sinorhizobium meliloti swimming colonies were imaged 48 h after incubation at 30° C. All other bacteria were imaged 24 h after incubation at 37° C.

### Surface Growth Inhibition

For intra-strain surface inhibition, exponential phase bacterial cultures were streaked in a square (2 cm×2 cm) onto a 100 mm round Petri dish using sterile cotton swabs. Plates were then spotted with 2 μL of 5×10⁷ CFU/mL of exponential phase culture arranged 0.5, 1.0, 1.5, and 2.0 cm (Left, Top, Right, Bottom, respectively) from the edge of the square streak, with a 5th inoculant added to the center region of the square streak. Bacterial cultures were spotted at the same time point (+0 h) or at 24, 48, or 72 h after incubation of the square streak. All plates were incubated at 37° C. in a sealed container and imaged daily for 5 days. Similarly, E. coli BW25113 streaked as the producer strain in a 3×3 grid pattern on a 100 mm square Petri dish for broad spectrum inhibition assay.

### Cfu Comparison of Inoculants Spotted in the Square Streak

Similar to the method described above, E. coli BW25113 was streaked onto LB in a square pattern and then was spotted in the center 24- or 48-h after incubation of the streak. An inoculant (2 μL of 5×10⁷ CFU/mL) was also spotted on cell-free agar containing only 0.85% NaCl as a control. Spotted plates were incubated at 37° C. for 24 h. CFU was determined by cutting out a thin layer where the inoculant was spotted, placing it in 100 μL of LB broth, and vortexing it rigorously for 10 s. 50 μL of the suspension was removed, serially diluted, and plated onto LB 1.5% agar and incubated at 37° C. for 24 h. Mean CFU for each condition was calculated from 3 replicates. Statistical analysis was done by using 1-way ANOVA followed by Tukey's test using GraphPad Prism.

### Live/Dead Staining and Confocal Imaging

Integrity of E. coli BW25113 cells inhibited by other E. coli BW25113 inside a square steak was determined using Live/Dead BacLight Bacterial Viability Kit (L13152, Sigma) with some modification. After a 24-h incubation, E. coli BW25113 cells (2 μL of 5×10⁷ CFU/mL) were spotted in the center of the square streak and incubated at 37° C. for 90 min or onto fresh LB 0.8% agar as a control. The spotted E. coli BW25113 cells were stained by adding 10 μL of 2× concentration of SYTO9 (12 μM) and Propidium Iodide (60 μM) on the spotted bacterial cells and incubated at 21° C. in the dark for 15 min. This process was repeated one more time to ensure sufficient staining. The agar with stained inoculant was cut from the plate and placed inverted in a glass bottom petri dish (P50G-1.5-30-F, Matek) so that spotted bacterial cells are facing the glass bottom. Bacterial cells were imaged on a Leica SP5 laser confocal microscope using a 63× water immersion objective (HCX PLO APO CS 1.20 NA), 3× digital zoom, and sequential scanning at 488 nm/500-530 nm (excitation/emission) for SYTO9 and 543 nm/604-700 nm Propidium Iodide.

### Image Analysis

Confocal images of live/dead staining were analyzed using Cell Profiler version 4.2.5 (cellprofiler.org) to determine the percentage of PI positive cells. Images were converted to grayscale using the “ColorToGray” module and cells were counted using the “IdentifyPrimaryObjects” module. Object identification parameters used advanced settings with the lower threshold of object diameter set to the measured pixel width of the smallest bacterial cell. Cells outside of this range and near the border of the image were discarded. A global threshold strategy utilizing the Otsu method with 3 classes was employed. this process was repeated with the “ColorToGray” module employed to split the RGB image into independent channels and only converting objects with a red signal into grayscale, followed by cell counting with the parameters set above. This ensures any cell with red signal (PI stain) will be counted, as cells can contain a mixture of both dyes to indicate cells with varying degrees of membrane damage. Cell counts from 3 fields of view for each condition were obtained, and percent PI positive cells was calculated.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of screening for an antibiotic, the method comprising sequential steps of:
streaking a first bacterial sample to form surrounded internal space on a supporting media, thereby forming an initial streak;
waiting at least 1 day;
spotting a second bacterial sample on the supporting media in a center of the initial streak, thereby defining a spot location;
waiting at least 2 days;
harvesting a sample at a location between the spot location and an edge of the initial streak, thereby producing a harvested sample; and
isolating an antibiotic from the harvested bacteria sample.

2. The method as recited in claim 1, wherein the surrounded internal space is formed by a square streaking pattern.

3. The method as recited in claim 1, wherein the surrounded internal space is formed by a circular streaking pattern.

4. The method as recited in claim 1, wherein the waiting at least 1 day, comprises incubating at a temperature from 30-37° C.

5. The method as recited in claim 1, wherein the waiting at least 2 days, comprises incubating at a temperature from 30-37° C.

6. The method as recited in claim 1, wherein the waiting at least 1 day, comprises incubating at a temperature from 30-37° C. and the waiting at least 2 days, comprises incubating at a temperature from 30-37° C.

7. The method as recited in claim 1, wherein the first bacteria sample and the second bacteria sample are the same species of bacteria.

8. The method as recited in claim 1, wherein the first bacteria sample and the second bacteria sample are the same species and same strain of bacteria.

9. The method as recited in claim 1, wherein the first bacteria sample and the second bacteria sample are the different species of bacteria.

10. The method as recited in claim 1, wherein the supporting media is agar.

11. The method as recited in claim 1, wherein the supporting media is agarose.

12. The method as recited in claim 1, wherein the supporting media is a porous media with liquid growth medium.

13. A method of screening for an antibiotic, the method comprising sequential steps of:
streaking a first bacteria sample in a pattern on a supporting media, wherein the pattern has a pattern edge;
waiting at least 1 day;
spotting a second bacterial sample on the supporting media at a distance of at least 0.5 cm but less than 2.0 cm from an edge of the pattern edge;
waiting at least 2 days;
harvesting a sample at a location between the spot location and the pattern edge, thereby producing a harvested sample; and
isolating an antibiotic from the harvested bacteria sample.
